(19) United States
(12) Patent Application Publication
(10) Pub. No.: US 2025/0034517 A1
(43) Pub. Date: Jan. 30, 2025

(54) Substrate Assembly, Cell Culture Method, Bioreactor and Cultured Meat Product

(71) Applicant: Ivy Farm Technologies Limited, Oxfordshire (GB)

(72) Inventors: Simon William Partridge, Oxford (GB); Andrew Tan-Mulligan, Oxford (GB); Kiruthika Lawton, Oxford (GB); Robin Nyland, Oxford (GB)

(21) Appl. No.: 18/837,172

(22) PCT Filed: Feb. 8, 2023
(86) PCT No.: PCT/GB2023/050288

§ 371 Date: Aug. 8, 2024

(30) Foreign Application Priority Data
Feb. 8, 2022 (GB) 2201603.4

(51) Int. Cl. C12N 5/00; A23L 13/00; C12N 5/077

(52) U.S. Cl. CPC C12N 5/0068; A23L 13/00; C12N 5/0653; C12N 5/0658; C12N 2533/18; C12N 2533/74

(57) ABSTRACT

There is provided a substrate assembly for culturing cells, wherein the substrate assembly comprises: a plurality of edible fibres, wherein each fibre has an internal channel running along its length; a first support at a first end of the plurality of edible fibres; a second support at a second end of the plurality of edible fibres; wherein at least one of the first support and second support allows fluid communication across the support into the internal channels. Also provided is a method of culturing cells.

### FIELD

The present invention relates to a substrate assembly for culturing cells. The present invention further relates to a method of culturing cells. The present invention also relates to a bioreactor system.

### BACKGROUND OF THE INVENTION

There are concerns about the sustainability of traditional farming practices relating to the rearing of livestock. These traditional farming practices are energy intensive, land intensive and utilise a large amount of antibiotics. A possible approach for addressing these concerns is culturing of meat in a bioreactor. This requires far less energy and land and is a desirable approach for feeding the world's growing population.

However, the success of meat culture depends on the commercial viability of the culture process. The present invention provides means for increasing meat culture viability and efficiency.

### SUMMARY OF THE INVENTION

The present invention provides a substrate assembly for culturing cells, wherein the substrate assembly comprises: a plurality of edible fibres, wherein each fibre has an internal channel running along its length; a first support at a first end of the plurality of edible fibres; a second support at a second end of the plurality of edible fibres; wherein at least one of the first support and second support allows fluid communication across the support into the internal channels.

This edible substrate assembly provides a reduced risk of contamination with non-edible materials when incorporating the resulting cell culture into a final product that is intended to be eaten. In particular, the cultured meat on the substrate can be incorporated at least partially into the meat product for consumption along with the edible substrate itself. This vastly simplifies the processing of the product.

The cultured cells may be removed from the bioreactor and used directly as a replacement for livestock-derived meat in a meat product for consumption using suitable food production techniques as are well known to the skilled person. For example, the cultured cells may be simply added into a mixture intended to form meatballs or sausages as a replacement or addition to livestock-derived meat, and the mixture then processed into the meat product using conventional techniques.

The present invention can be generally used for the culture of cells, in particular adherent cells (cells that grow on a substrate, also known as anchorage-dependent cells). The invention is particularly advantageous for culturing muscle and/or fat cells for the production of meat for consumption, e.g. human consumption. Thus, the cells introduced (seeded) into the bioreactor may be capable of differentiating into myocytes (including myotubes) and/or adipocytes. In particular, the cells introduced into the bioreactor may be stem cells, progenitor cells, or precursor cells. Exemplary cells include embryonic stem cells (ESCs), induced pluripotent stem cells (iPSCs), mesenchymal stem cells (MSCs), muscle stem cells (muscle satellite cells), myoblasts, pre-adipocytes, or combinations thereof. Myoblasts are particularly preferred. The cells introduced into the bioreactor may be primary cells or cell lines. Exemplary cell lines include Chinese Hamster Ovary (CHO) cells and C2C12 cells (a myoblast subclone).

During cell culture in the bioreactor, the cells or a portion of the cells may differentiate into myocytes (including myotubes) and/or adipocytes (including white fat cells and brown fat cells). Myocytes (including myotubes) are particularly preferred. Thus, the invention may be used for differentiating cells.

### DETAILED DESCRIPTION OF THE INVENTION

Cells for use in the invention may be of any animal origin. However, typically, the cells are not human cells. In particular, preferably, the cells are not produced through the destruction of human embryos. The cells are typically cells of non-human animals, such as non-human mammals, birds, fish, crustaceans, molluscs, reptiles, amphibians, or insects. Exemplary non-human mammals include those in the genera Bovinae, Camelidae, Canidae, Caprae, Cervidae, Felidae, Equidae, Lagomorphs, Macropodidae, Oves, Rodents, or Suidae. The cells may be cells of livestock or poultry. The cells may be porcine, bovine, ovine, caprine, avine, or piscine.

The substrate includes a plurality of fibres. These fibres may be edible, although this is not essential for all embodiments of the invention. Edible fibres are those which can be safely eaten by humans and/or are considered safe for eating by humans, in line with the common understanding of edible products. Edible fibres may comprise substantially, consist essentially of, or consist of materials designated Generally Recognized as Safe (GRAS) by the US Food and Drug Administration (FDA), in particular under sections 201 (s) and 409 of the Federal Food, Drug, and Cosmetic Act (FDCA) and/or materials which comply with the Food Chemicals Codex (FCC). The edible fibres may be digestible by humans. The edible fibres may have nutritional value for humans.

Each of the fibres has an internal channel running along its length. In this manner, fluid (preferably comprising cells) is capable of flowing down the internal channel of each of the fibres. The fibres may have an outer diameter of 0.5 millimetres or greater, preferably one millimetre or greater. The outer diameter may be two centimetres or less, preferably 1.5 millimetres or less. The inner diameter of the fibres may be 0.2 millimetres or more, preferably 0.4 millimetres or more. The inner diameter of the fibres may be 1.6 centimetres or less, preferably 0.8 centimetres or less. These diameters are beneficial for stability of the fibres.

The fibres may be any suitable length. In particular, the fibres are preferably of a length that efficiently uses the length of the bioreactor into which they will be placed. For example, the plurality of fibres may each be at least 0.2 metres long in length, preferably at least 0.5 metres in length. For example, the plurality of fibres may each be less than 5 metres in length, preferably less than 3 metres in length. These lengths are beneficial for stability of the fibres.

The plurality of fibres may be substantially parallel. This assists with the attachment, proliferation, and differentiation of cells that benefit from alignment within the substrate. For example, it can encourage myogenesis (myotube formation). Substantially parallel edible fibres also assist in creating a product which mimics the texture of natural meat.

The plurality of fibres are supported at their ends in order to hold the fibres in the desired position when they are in the bioreactor. Preferably, the first support and the second support hold the plurality of fibres so that they extend between the supports in a straight line when in place within the bioreactor. Alternatively, the first support and the second support may hold the plurality of fibres so they extend as a loop, i.e. the plurality of fibres extends away from the support in a substantially first direction and then loop round to return in the general direction of the first support. This can be easily achieved by having the first support regions, that support the first ends, integrally formed with the second support regions, that support the second ends. In other words, the first and second supports can be formed as one component. In contrast, the first and second supports can be formed as separate components. In order to form the hollow fibres into a loop, the first and second supports may be positioned adjacent each other when placed in the bioreactor. In either case, the flow of any medium fluid can be directed to enter the first ends and exit the second ends by any suitable means.

Each support may be configured in the same or in a different manner. One or both of the supports may be formed from a resin, such as an epoxy resin. In this manner, the ends of the fibres can be placed into the epoxy resin which is subsequently cured to hold the fibres in place.

At least one of the first support and second support allows fluid communication across the support and into the internal channels of the plurality of fibres. In this manner, the support facilitates material, such as cells and cell culture medium, being introduced into the internal channel of the fibres. This allows the possibility of growing cells on the internal and external surfaces of the fibres, which increases the surface area for adherent cells to grow on and thus increases the possible yield of cells from the substrate. In a simple way, the support may allow fluid communication across itself and into the internal channels by having exposed openings into the channels on the face of the support. Both supports may allow fluid communication across themselves and into the internal channels. This advantageously allows ease of access to the internal channel for fluid and also allows fluid to flow through the fibres from one end of the internal channels to the other.

The plurality of fibres may comprise alginate. Alginate is edible as it is not harmful when consumed and is used in relation to food for consumption. Although it is not necessary to disassociate the cells or all of the cells from an edible substrate, it may be desirable to remove or reduce the amount of edible substrate in the final product, for example to improve texture. The use of alginate increases the ease of disassociating cells from the substrate after they have been cultured on the substrate. In particular, disassociation of cells can be achieved by using alginate-lyase to break down the alginate. Any appropriate digestion enzyme can be utilised depending on the material used to form the (edible) fibres.

The plurality of fibres may comprise one or more polysaccharides as an edible material.

The fibres may comprise peptide conjugation. The peptide conjugation may be operable to affect cell adhesion, proliferation, and/or differentiation. The peptide conjugation may be formed through reaction with a carboxyl group of the fibre material, such as a carboxyl group of alginate. For example, the peptide conjugation may comprise 1-ethyl-(dimethylaminopropyl) carbodiimide (EDC) and/or N-hydroxysuccinimide (NHS) peptide conjugation, such as with Gly-Arg-Gly-Asp-Tyr (GRGDY) and/or Tyr-IIe-Gly-Ser-Arg (YIGSR).

The present invention also relates to a method of culturing cells, wherein the method comprises the following steps: obtaining the substrate assembly described herein; and culturing cells on the substrate assembly in a bioreactor.

The bioreactor that can be used with the present invention is any suitable bioreactor for culturing adherent cells. In general terms, a bioreactor is a vessel that can support the maintenance and growth of cells. This is also referred to as the culture of cells.

The bioreactor houses the substrate along with cell culture medium, and optionally cells. The cell culture medium may contain nutrients and growth factors within a fluid that supports the maintenance, proliferation, and growth of the cells. The cell culture medium may contain factors promoting cell differentiation. An exemplary culture medium is Dulbecco's Modified Eagle Medium (DMEM). Cell culture medium for use in the invention may comprise serum or may not comprise serum. The cells that are adhered on the substrate are submerged in the cell culture medium. The cell culture medium may be moved around the bioreactor to facilitate the growth of the cells.

A particularly preferred approach is to use perfusion of the cell culture medium. Perfusion refers to the introduction of medium into the bioreactor while removing medium (e.g. comprising lactic acid) from the bioreactor. It is particularly preferred that the new medium is introduced at the bottom of the bioreactor and the spent medium is removed from the top of the bioreactor. The new medium can be introduced by being pumped into the bioreactor. The spent medium may be removed by being pumped out of the bioreactor. The introduced medium may be fresh medium, in the sense it has not been previously introduced into the bioreactor and the removed medium can be spent medium, in the sense that it has been exposed to growing cells. However, the perfusion approach encompasses recirculating, at least part of, the medium through the bioreactor.

The bioreactor can regulate the temperature of the medium within which the cells are growing. This allows optimization of the cell growth.

The bioreactor may be any suitable size for culturing the cells. Accordingly, the bioreactor may have a capacity of at least 1 litre, or at least 2 litres, or at least 3 litres. A bioreactor system may be employed that contains a plurality of bioreactors, where each bioreactor is used to culture cells on its own substrate.

The substrate assembly may be incorporated into the bioreactor such that fluid can be introduced into the internal channels of the fibres and fluid may be introduced around the fibres so as to contact the external surfaces of the fibres. It is particularly preferred that the substrate assembly, when placed in the bioreactor, prevents fluid that is introduced into the internal channels from being introduced directly into the region contacting the external surfaces of the fibres and conversely that fluid introduced into the region that contacts the external surfaces is prevented from going directly into the internal channels. The reference to directly allows for the possibility that the media may exchange indirectly through the potentially porous walls of the fibres. Preferably the walls are not porous to cells, even if they are porous to fluids.

Accordingly, at least one of the first support and second support that allows fluid communication across the support into the internal channels also prevents fluid communication across the support to the external surfaces of the plurality of fibres. This can be achieved by the support being of the form of a cured resin where the openings to the channel are exposed on the face of the support while the resin seals the regions between the fibres.

The fibres may be produced using an extrusion step. The use of extrusion can result in orientation features on the fibres such as surface grooves that, along with the alignment of all of the fibres, supports the alignment of the cells when they adhere to the surface of the fibres. This can encourage cell alignment and thus myogenesis (myotube formation). This can also improve the ability of the product to mimic the texture of natural meat.

It should be understood that each of the plurality of fibres extends continuously from the first support to the second support.

The fibres may be formed as an initial continuous fibre from a fibre drawing process such as the extrusion described herein. This continuous fibre may be wound around a collector such that the fibre is oriented to run along the length of the collector and then turn back on itself as it goes around the end of the collector and runs back in the opposite direction. The continuous fibre can be wound numerous times around the collector. In this manner, a plurality of parallel straight sections of fibre can be readily produced. The points at which the fibre changes direction may then be cut to produce the plurality of discrete fibres. A specific approach would be to cure resin onto either end of this wound continuous fibre and then to cut the resin such as to cut the individual fibre into the plurality of distinct fibres with exposed internal channel openings on the cut resin surface.

The fibre may comprise two or more materials. This can be achieved by coextruding the fibres such that the two materials are extruded at the same time. For example, alginate may be used to form an outer surface of the fibre while calcium chloride is used to form the inner surface of the fibre. The use of two materials allows one material to be included that modifies the other material. For example, the presence of calcium chloride acts as a cross-linking agent for alginate. Accordingly, the second material may be a cross-linking agent for the first material, i.e. an agent that elicits bonding in the first material, such as ionic cross-linking or covalent crosslinking. The cross-linking agent may be provided in solution as the second material, such as in glycerol. It is particularly preferred that the solution is thixotropic, such as xanthan gum. This allows the solution to be readily extruded but provides sufficient support to the fibre after the extrusion has occurred.

The fibres may be subjected to a freeze-drying step during their production. The freeze-drying step has been found to be particularly useful when the fibres comprise alginate. The freeze-drying step can help to improve the mechanical properties of the fibres. The freeze-drying step can also be used to adjust the porosity of the final fibre material.

The mechanical properties of the fibres may also be adjusted by including additives into the fibre composition. For example, plasticisers or viscosity modifiers may be added to help the fibres maintain the required shape with the presence of the internal channel. For example, the viscosity modifier may comprise xanthan gum and/or agarose.

The viscosity modifier may comprise a powder, such as a powder that is soluble in the carrier liquid of the solution of the second material.

The viscosity modifier may have a melting temperature of up to 65° C., such as up to 55° C. or up to 50° C. Advantageously, a melting temperature within these parameters may allow for the structure of the inner channel to be maintained while the internal material is dissolved, and optionally replaced, using a non-contact method. The ability to change the internal material using a non-contact method may be advantageous compared to a mechanical extraction method because it can permit for operation under sterile conditions and may be more scalable.

The internal channel may also be maintained by forming the fibre so that the internal channel is filled with a fluid initially. This can be achieved by extruding the fibre into a bath of the relevant liquid. The liquid in the internal channel may be non-aqueous, such as ethanol. This supports the shape of the fibre. The initial fluid being a non-aqueous fluid, can assist with any freeze-drying step. This fluid can be subsequently removed when the substrate assembly is placed in the bioreactor and the fluid that is used for culturing cells is introduced into the channel.

Singular encompasses plural and vice versa. For example, although reference is made herein to “a” particulate substrate, “a” flow distributor, and the like, one or more of each of these and any other components can be used.

The terms “comprising” and “comprises” as used herein are synonymous with “including”, “includes” or “containing”, “contains”, and are inclusive or open-ended and do not exclude additional, non-recited members, elements or method steps.

Additionally, although the present invention has been described in terms of “comprising”, the invention as detailed herein may also be described as “consisting essentially of” or “consisting of”.

Although the present invention has been described in terms of “obtainable by”, the associated features of the present invention detailed herein may also be independently described as “obtained by”.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

Where ranges are provided in relation to a genus, each range may also apply additionally and independently to any one or more of the listed species of that genus.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

The present invention will be described in relation to the following Figures and examples.

### FIGURES

FIG. 1 depicts a schematic overview of the manufacture of the fibres;

FIG. 2 shows brightfield images of cell growth on the alginate, alginate-GRGDS and TCP control examples.

### EXAMPLES

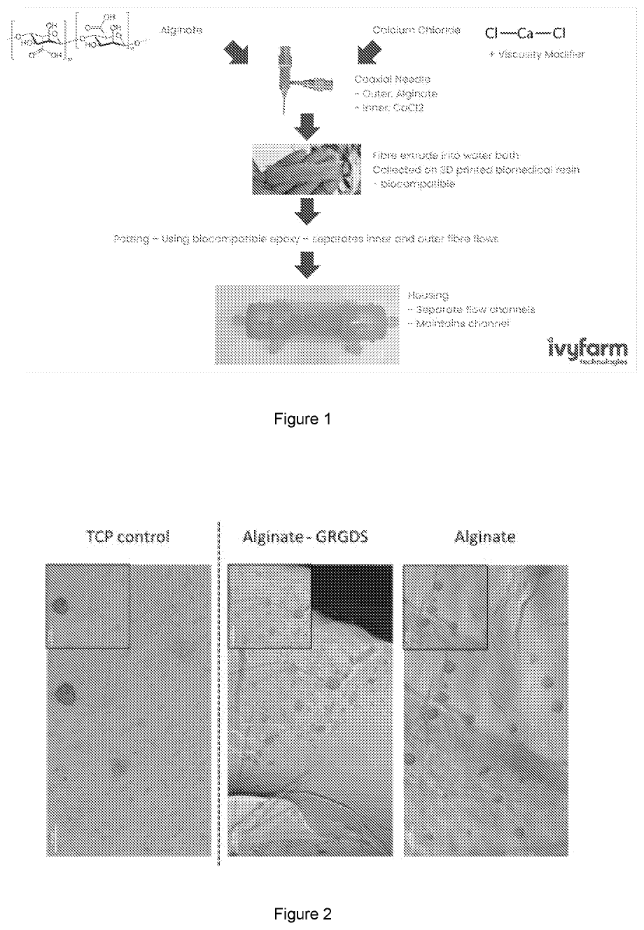

The manufacture process of the invention was carried out as detailed in FIG. 1. Medium viscosity alginate at 2 wt % dissolved in distilled water was used to form the fibres along with calcium chloride at 0.1M with 1 wt % temperature sensitive agarose dissolved in distilled water. The alginate solution is extruded through the outer channel of a co-axial needle while the calcium chloride and the agarose non-water-based viscosity modifier is extruded through the inner channel. The coaxial needle has a 25 inner gauge and an 18 outer gauge. This equates to 1.2 mm and 0.5 mm in diameter respectively.

The solution is extruded into an ethanol bath and collected on a rotating collector frame. The collector has a length of 20 cm and a width of 5 cm. The material is allowed to dry and then potted at both ends with biomedical resin. The potting material used is bio-compatible epoxy. The fibres are dipped in a tube with the epoxy. The epoxy is left to cure overnight. After the epoxy is cured, the ends are exposed by buzzsaw.

The potted ends are thus cut in half to expose the hollow inner channel, thus creating a separation of flow between the inner and outer channels. The completed substrate assembly is finally placed within a bioreactor container.

The following examples compare cell growth on substrates for use in the present invention against a control.

The substrates tested were:
Crosslinked alginate-GRGDSP
Crosslinked alginate unconjugated
TCP control

The materials used were as follows:
Alginic acid-medium viscosity-A2033-250G #SLCF1476
Calcium chloride 0.3M
1-ethyl-(dimethylaminopropyl) carbodiimide (EDC) (50 mg EDC/g alginate) CAS 25952-53-8
N-hydroxysuccinimide (NHS) (28 mg NHS/g alginate) CAS 6066-82-6 Peptide (GRGDSP) (1 mg peptide/g alginate)
MES buffer 0.1M pH 6.5

A peptide conjugation of alginate solution was prepared as follows:
1. An alginate 1% (wt/v) solution was prepared in a MES buffer.
2. EDC and NHS solutions were prepared in MES buffer.
3. The NHS solution was added to the alginate solution followed by addition of the EDC solution. The tube containing the mixture was covered with foil to protect it from light and placed on a rocker for 20 min at room temperature.
4. The peptide was solubilised in PBS (without Ca and Mg) and the appropriate amounts of the peptide were then added into the solution mixture.
The solution mixture was allowed to conjugate at room temperature under gentle agitation for 18 to 20 hours.
5. The reaction mixture was dialysed for 5 days against excess of deionized water, to remove residual reactants.

An alginate solution was prepared as follows:
1. An alginate 1% (wt/v) solution was prepared in a MES buffer.

Alginate films were prepared as follows:
500 ul of each solution (the peptide conjugation of alginate solution and the alginate solution) was pipetted into separate wells.
The plate was placed in an oven at 40 C for 18 hours to dry the alginate-GRGDS and alginate films.
The dried films were then UV sterilised for 1 hour prior to crosslinking with 20 mM Calcium Chloride for an additional 1 hour.
After crosslinking, the films were washed with excess medium twice (5 minute washes each).

Media info:
DMEM with 20% FBS, 1% L-Glutamine, 1% Pencillan and Streptomycin and FGF-2 (5 ng/ml).

Cells were cultured on each of the peptide conjugation of alginate film, the alginate film and the TCP control as follows:
A cell seeding density of 8,000 cells per cm² was used.
The cells were cultured for 96 hours.
The films were picked up with forceps and placed into a new well containing PBS to avoid imaging cells that had adhered to well bottom.
Images were taken in brightfield to visualize cells and qualitatively assess cell attachment and morphology.

A higher number of cells (per field of view) was observed on alginate-GRGDS films. These cells had an elongated morphology. It was also surprisingly observed that cells had also adhered and elongated on the non-treated alginate film.

1. A substrate assembly for culturing cells, wherein the substrate assembly comprises:
a plurality of edible fibres, wherein each fibre has an internal channel running along its length;
a first support at a first end of the plurality of edible fibres;
a second support at a second end of the plurality of edible fibres;
wherein at least one of the first support and second support allows fluid communication across the support into the internal channels.

2. The substrate assembly of claim 1, wherein the plurality of fibres comprise alginate.

3. The substrate assembly of claim 1, wherein both the first support and second support allow fluid communication across the support into the internal channels, such that fluid can flow across the first support, through the internal channels, and then across the second support.

4. A method of culturing cells, wherein the method comprises the following steps:
obtaining the substrate assembly of claim 1; and
culturing cells on the substrate assembly in a bioreactor.

5. The method of claim 4, wherein the at least one of the first support and second support that allows fluid communication across the support into the internal channels also prevents fluid communication across the support to the external surfaces of the plurality of fibres.

6. The method of claim 1, wherein the step of obtaining the substrate assembly comprises an extrusion step to obtain the plurality of fibres.

7. The method of claim 1, wherein the step of obtaining the substrate assembly comprises a co-extrusion step to obtain the plurality of fibres, wherein each of the plurality of fibres has a first outer material and a second inner material.

8. The method of claim 7, wherein the first outer material comprises alginate and the second inner material comprises calcium chloride.

9. The method of claim 1, wherein the step of obtaining the substrate assembly comprises a freeze-drying step to obtain the plurality of fibres.

10. The method of claim 1, wherein the first type of cells and/or the second type of cells independently comprise myocytes, adipocytes, and/or cells capable of differentiating into myocytes and/or adipocytes.

11. The method of claim 1, wherein the plurality of fibres comprise alginate.

12. The method of claim 11, further comprising the step, after the step of culturing cells on the substrate, of disassociating the cells from the substrate by using alginate-lyase.

13. The method of claim 1, wherein both the first support and second support allow fluid communication across the support into the internal channels, such that fluid can flow across the first support, through the internal channels, and then across the second support.

14. A method of manufacturing the substrate assembly of claim 1, wherein the method comprises obtaining the plurality of edible fibres, and wherein the step of obtaining the plurality of edible fibres comprises an extrusion step to produce an extruded fibre.

15. The method of claim 14, wherein the extrusion step comprises co-extrusion such that each of the plurality of fibres has a first outer material and a second inner material.

16. The method of claim 15, wherein the first outer material comprises alginate and the second inner material comprises calcium chloride.

17. The method of claim 14, wherein the step of obtaining the plurality of edible fibres comprises a freeze-drying step.

18. The method of claim 4, further comprising a step of processing the cells into a meat product for consumption.

19. (canceled)

20. (canceled)


SEQUENCE LISTING (4 sequences)

SEQ ID NO: 1 - protein, 5 aa, synthetic construct
GRGDY

SEQ ID NO: 2 - protein, 5 aa, synthetic construct
YIGSR

SEQ ID NO: 3 - protein, 5 aa, synthetic construct
GRGDS

SEQ ID NO: 4 - protein, 6 aa, synthetic construct
GRGDSP